(19) United States
(12) Patent Application Publication
(10) Pub. No.: US 2025/0040921 A1
(43) Pub. Date: Feb. 6, 2025

(54) Knotless Suture Assemblies

(71) Applicant: Orthotek, LLC, Los Alamitos, CA (US)

(72) Inventor: Christopher Ninh, Irvine, CA (US)

(21) Appl. No.: 18/908,642

(22) Filed: Oct. 7, 2024

Related U.S. Application Data
(63) Continuation-in-part of application No. 18/593,772, filed on Mar. 1, 2024.
Application No. 18/593,772 is a continuation-in-part of application No. 17/736,015, filed on May 3, 2022, now Pat. No. 12,064,103.
Application No. 17/736,015 is a continuation of application No. 17/553,670, filed on Dec. 16, 2021, now Pat. No. 11,344,290.

(51) Int. Cl. A61B 17/04

(52) U.S. Cl. CPC A61B 17/0401; A61B 17/0485; A61B 17/0487; A61B 2017/0414

(57) ABSTRACT

A suture assembly comprises a suture anchor, a repair suture, and a shuttle suture. Each suture has a looped end and a tail end. The tail end of the repair suture and the looped end of the shuttle suture extend out one side of the proximal end of the anchor, while the looped end of the repair suture and the tail end of the shuttle suture extend out the opposite side of the anchor. The tail of the repair suture may have a proximal portion which is thicker than its end portion. The suture anchor may include a proximal bore receiving the repair suture and a distal bore receiving the shuttle suture. The distal bore may have a rear opening having a diameter approximately equal to the diameter of the proximal portion of the suture and a front opening that is larger than the rear opening.

### CROSS-REFERENCE TO RELATED APPLICATIONS

This application is a continuation-in-part of U.S. nonprovisional patent application Ser. No. 18/593,772, filed Mar. 1, 2024, which is a continuation-in-part of U.S. nonprovisional patent application Ser. No. 17/736,015, filed May 3, 2022, now U.S. Pat. No. 12,064,103B which is a continuation of U.S. nonprovisional patent application Ser. No. 17/553,670, filed Dec. 16, 2021, now U.S. Pat. No. 11,344,290B. The entire contents of each of the above applications are incorporated herein by reference.

### BACKGROUND OF THE INVENTION

### 1. Field of the Invention

The present disclosure relates in general to tissue repair surgery, and more particularly to knotless repair techniques and related devices.

### 2. Background Art

Labral tears can occur as a result of sports injuries and other types of trauma, and can also be caused by aging, as the labrum becomes brittle over time. Minor tears can be treated with medication, physical therapy, and rest, but more severe injuries require surgery.

Labral surgery can be open or arthroscopic, and can be performed using knotted or knotless techniques. A typical knotted repair technique involves loading a suture having two free ends into a suture anchor and implanting the suture anchor in the glenoid bone in a location on one side of the torn labrum. After one end of the suture is passed between the labrum and the glenoid, the two ends are tied together to create a knot securing the labrum to the bone. In a knotless repair technique, the suture is passed through the labrum first, and then its two ends are fed into the distal end of a suture anchor before the suture anchor is implanted in the bone. After the suture anchor has been implanted, the suture ends are held in place by an interference fit between the suture anchor and the bone.

Knotless labral repair techniques are generally preferred to knotted techniques, but they can be problematic since they allow relative movement between the labrum and the suture. In addition, suture slippage can occur if the interference fit between a knotless suture anchor and the surrounding bone is not sufficiently tight, as is frequently the case in patients having diminished bone density due to age or disease.

These and other problems are addressed by the techniques and devices summarized below, which can also be applied to a variety of other types of surgery including tissue repair, soft-tissue-to hard-tissue attachment, and hard-tissue-to-hard-tissue attachment.

### SUMMARY OF THE DISCLOSURE

In one aspect of the disclosure, a suture assembly includes an anchor body having a first side and a second side, a repair suture, and a shuttle suture. The repair suture has a looped end a straight linear tail, and is positioned such that its looped end extends in a proximal direction along the first side of the anchor body and its tail end extends in a proximal direction along the second side of the anchor body. The shuttle suture has a looped end and a tail end, and is positioned such that its looped end extends in a proximal direction along the second side of the anchor body, and its tail end extends in a proximal direction along the first side of the anchor body.

In one aspect of the disclosure, the loop of the repair suture is larger than the loop of the shuttle suture.

In one aspect of the disclosure, the tail of the repair suture has a proximal portion and an end portion, where the end portion is thinner than the proximal portion.

In another aspect of the disclosure, the loop and the tail have round cross-sections with predetermined diameters.

In another aspect of the disclosure, the cross-sectional diameter of the loop is less than the cross-sectional diameter of the proximal portion of the loop.

In another aspect of the disclosure, the tail comprises three sections, including the proximal portion, the end portion, and an intermediate portion between the proximal portion and the end portion.

In another aspect of the invention, the intermediate portion has a cross-sectional diameter that is greater than the cross-sectional diameter of the end portion and less than the cross-sectional diameter of the proximal portion.

In one aspect of the disclosure, the distal end of the suture anchor includes a suture engagement feature, and an intermediate portion of the repair suture engages the suture engagement feature.

In one aspect of the disclosure, the suture anchor includes an inner wall defining a cannulation, the suture engagement feature comprises a suture bridge extending across the cannulation.

In another aspect of the disclosure, the suture anchor is an all-suture anchor configured to expand radially outwardly and exert a contact force against surrounding bone when deployed.

In another aspect of the disclosure the suture anchor may be an all-suture anchor comprising a U-shaped collapsible tube formed of suture material. The tube defines an interior passage surrounded by an inner wall and includes a first side with a first proximal opening and a second side with a second proximal opening. The repair suture extends through the interior passage with its looped end extending out of the first proximal opening and its tail end extending out of the second proximal opening, while the shuttle suture extends in the opposite direction, with its looped end extending out of the second proximal opening and its tail end extending out of the first proximal opening. The inner wall of the interior passage functions as the suture engagement feature.

In one variation, a partition divides the interior passage of the all-suture anchor into a first channel and a second channel, wherein the repair suture extends through the first channel and the shuttle suture extends through the second channel.

In another variation, the suture anchor comprises a pair of U-shaped collapsible tubes positioned one on top of the other, wherein the repair suture extends through one of the tubes and the shuttle suture extends through the other tube.

In another aspect, the suture engagement feature comprises a bore extending through the suture anchor body, wherein both the repair suture and the shuttle suture pass through the bore. The cross-section of the bore may be round, oval, or teardrop shaped.

In one variation, the suture engagement feature comprises a pair of bores, wherein the repair suture extends through one bore, and the shuttle suture extends through the other bore. The two bores may be the same size as one another, or one may be larger, with the repair suture passing through the larger bore. The cross-section of each bore may be round, oval, or teardrop shaped.

In some double-bore variations, the two bores include a proximal bore and a distal bore, wherein the distal bore includes a rear opening with a dimension selected to create an interference fit with the thickened portion of the repair suture

In another variation, the suture anchor may include an inner cannulation with two suture bridges extending across the cannulation, wherein the repair suture engages one suture bridge and the shuttle suture engages the other suture bridge.

In another aspect of the disclosure, a method of attaching tissue to bone comprises 1) loading a repair suture having a looped end and a tail into a suture anchor in such a way that the looped end of the repair suture extends in a proximal direction along one side of the anchor body and the tail end of the repair suture extends in a proximal direction along the other side of the anchor body; 2) implanting the suture anchor into a bone below and to one side of the tissue; 3) passing the tail of the repair suture between a detached tissue and a bone; 4) passing the tail of the repair suture back over the detached tissue and through the loop of the repair suture to create a luggage tag construct; and 5) locking the tail of the repair suture against the suture engagement feature to prevent slippage of the suture.

In one aspect of the method, the suture anchor includes an inner wall including a cannulation, the suture engagement feature comprises a suture bridge extending across the cannulation, and the tail of the repair suture includes a thickened portion. In this aspect, the step of locking the tail of the repair suture comprises capturing the repair suture in an interference fit between the suture bridge and the inner wall.

In another aspect, the method further includes loading the suture anchor with a shuttle suture having a looped end and a tail, wherein the shuttle suture extends in a direction opposite the repair suture, such that its looped end extends proximally along the second side of the anchor body, and its tail end extends proximally along the first side of the anchor body. In this aspect, the suture engagement feature comprises a first bore extending through the suture anchor body, and a second bore extending through the suture anchor body, and the repair suture extends through the first bore and the shuttle suture passes through the second bore.

### BRIEF DESCRIPTION OF THE DRAWINGS

FIGS. 1A-E illustrate a prior art method of securing a torn shoulder labrum to a glenoid bone.

FIG. 2A is a front view of a labral anchor in a pre-deployed configuration.

FIG. 2B is a longitudinal sectional view of FIG. 2A.

FIG. 3 is a longitudinal sectional view showing the labral anchor of FIGS. 2A and 2B in a partially deployed configuration.

FIG. 4A is a front view of the labral anchor of FIGS. 2A and B in a fully deployed configuration.

FIG. 4B is a longitudinal sectional view of FIG. 4A.

FIG. 5 shows a repair suture.

FIGS. 6A-F represent examples of suture assemblies.

FIGS. 7-12 illustrate a method of securing a torn shoulder labrum to a glenoid bone using the suture assembly of FIG. 6A.

FIGS. 13A-E illustrate a method of securing tissue to bone using the suture assembly of FIG. 6D.

FIGS. 14A-C represent examples of single-bore labral suture anchors.

FIGS. 15A-F represent examples of double-bore labral suture anchor tips.

FIGS. 16A-C represent examples of single-bore lateral row suture anchor tips.

FIGS. 17A-F represent examples of double-bore lateral row suture anchor tips.

FIG. 18A is a side view of an alternate repair suture

FIG. 18B is an enlarged view of Detail A of FIG. 18A.

FIG. 18C is a sectional view taken through line C-C pf FIG. 18B.

FIG. 18D is an enlarged view of Detail D of FIG. 18A.

FIG. 18E is an enlarged view of Detail E of FIG. 18A.

FIG. 18F is a sectional view taken through line F-F of FIG. 18A.

FIG. 19A is a front view of another example of a double-bore labral suture anchor tip.

FIG. 19B is a cross-sectional view of the double-bore labral suture anchor tip of FIG. 18A.

FIG. 20A is a front view of another example of a of double-bore lateral row suture anchor tip.

FIG. 20B is a cross-sectional view of the lateral row suture anchor tip of FIG. 19A.

### DETAILED DESCRIPTION OF THE PREFERRED EMBODIMENT(S)

As required, detailed embodiments of the present invention are disclosed herein; however, it is to be understood that the disclosed embodiments are merely exemplary of the invention that may be embodied in various and alternative forms. The figures are not necessarily to scale; some features may be exaggerated or minimized to show details of particular components. Therefore, specific structural and functional details disclosed herein are not to be interpreted as limiting, but merely as a representative basis for teaching one skilled in the art to variously employ the present invention.

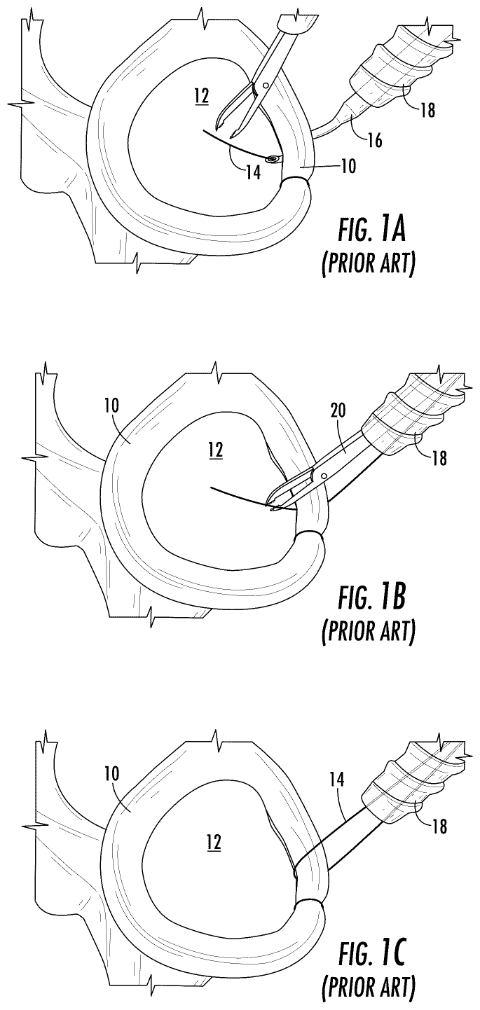

FIGS. 1A-E illustrate a conventional, knotless, suture-first technique for securing a torn shoulder labrum 10 to a glenoid bone 12. In FIG. 1A, the distal end of a conventional straight suture 14 is passed between the labrum 10 and the bone 12 using a suture passing device 16 that has been extended through a posterior cannula 18. After the suture 14 is fed into the joint, the suture passing device 16 is withdrawn, and a grasping instrument 20 is extended through the posterior cannula 18 to grab the suture 14 as shown in FIG. 1B. The distal end of the suture 14 is then drawn into the posterior cannula 18, as shown in FIG. 1C. Next, a socket 22 is formed in the bone 12 using a drill 24 or similar tool extended through the posterior cannular 18, as shown in FIG. 1D. Finally, the distal end of the suture 14 is fed through the distal end 26 of a suture anchor 28, which is then inserted into the bone socket 22, as shown in FIG. 1E. Typically, this procedure is repeated two more times, so that the labrum 10 is secured to the bone 12 at three locations.

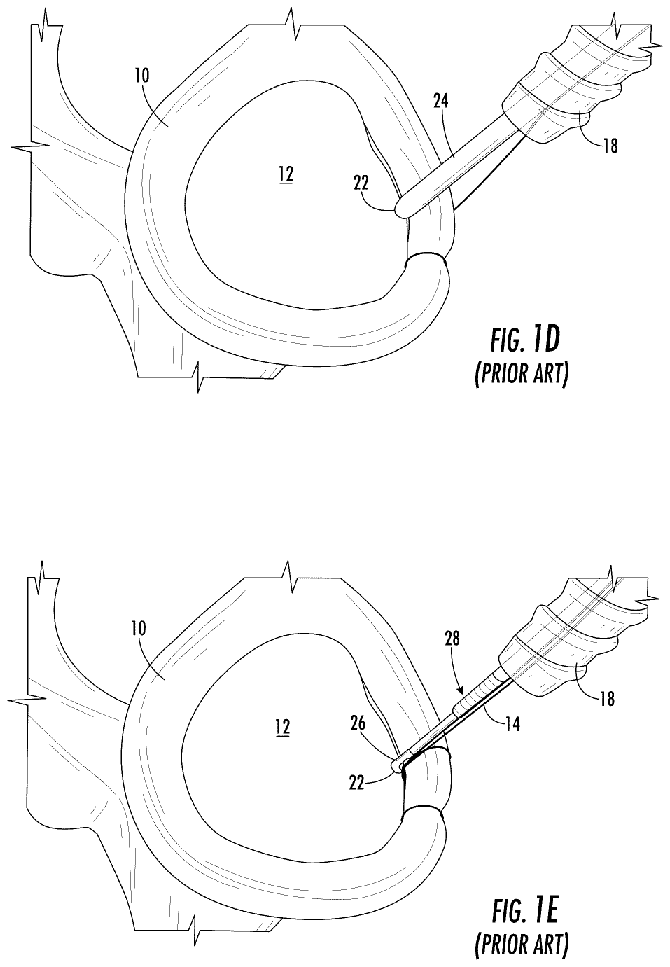

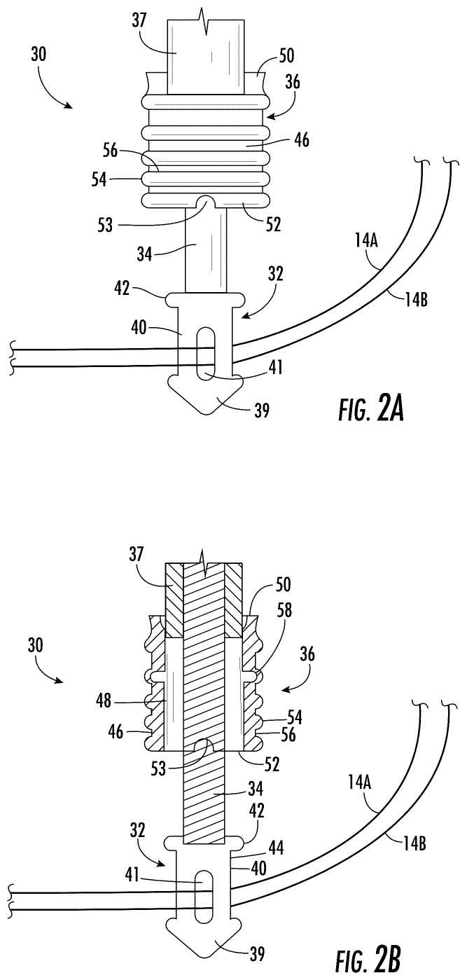

FIGS. 2A and B show a suture anchor 30 usable in the method of FIGS. 1A-E or other conventional knotless, suture-first, soft tissue-to-bone reattachment methods. The suture anchor 30 comprises a distal tip 32 detachably secured to a distal end of an inner driver shaft 34 that extends through the interior of a proximal anchor body 36. An outer driver shaft 37 is mounted for sliding movement relative to the inner driver shaft 34. The proximal anchor body 36 is detachably secured to and slidable with the distal end of the outer driver shaft 37.

The distal tip 32 includes an enlarged, rounded distal portion 39 and a proximally extending neck 40 An elongated eyelet 41 formed in the neck 40 is configured to receive the ends of sutures 14A, B. An annular bead 42 extends around the exterior of the proximal end 44 of the neck 38.

The proximal anchor body 36 is tubular in configuration and includes an exterior wall 46, an interior wall 48, a proximal end 50, and a distal end 52 having a semicircular notch 53. The exterior wall 46 comprises a plurality of rounded annular ribs 54 separated by rounded annular channels 56. An annular groove 58 is formed in the interior wall 48 at the proximal end 50.

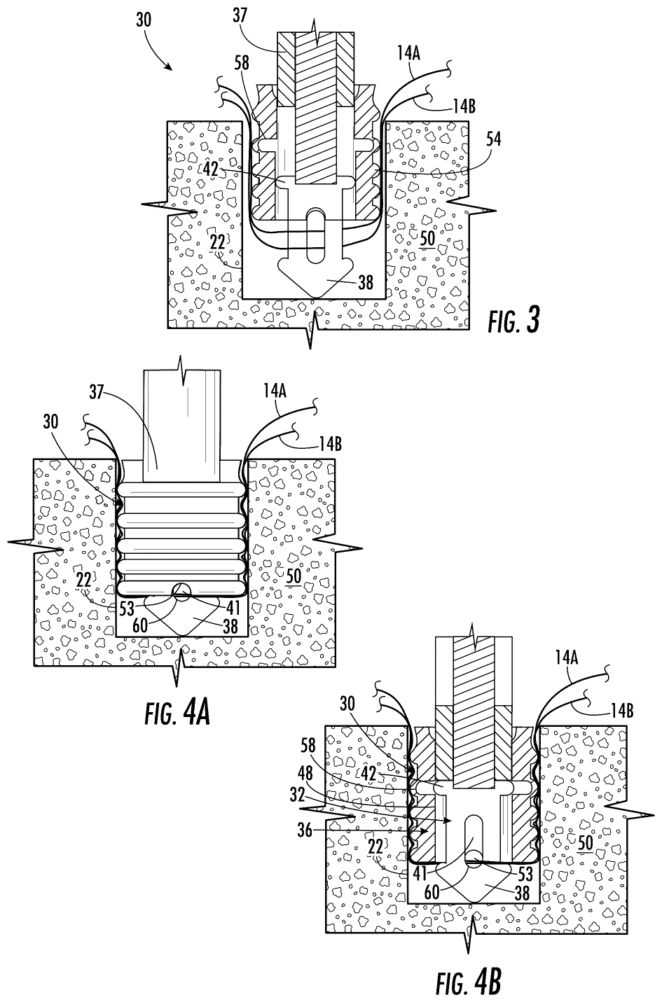

To secure the sutures 14A, B within a bone 50, a surgeon inserts the distal tip 38 of the suture anchor 30 into a pre-drilled bone socket 22, and pushes down on the outer driver shaft 37, causing the outer driver shaft 37 and proximal anchor body 36 to move together toward the distal tip 38, as shown in FIG. 3. Downward movement of the outer driver shaft 37 and proximal anchor body 36 continues until the annular groove 58 at the proximal end 50 of the proximal anchor body 36 snaps over the annular bead 42 on the neck 40 of the distal tip 36, locking the proximal anchor body 36 and the distal tip 36 together, as shown in FIGS. 4A and 4B. At this point, the outer and inner driver shafts 34, 37 are withdrawn, and the anchor 30 remains in the bone 50, with sutures 14A, B trapped between the ribs 54 of the proximal anchor body 37 and the walls of the bone socket 22, as well as between the distal end 60 of the eyelet 40 and the proximal end of the semicircular notch 53.

FIG. 5 shows a repair suture 114 usable in an alternate method for securing torn soft tissue such as a shoulder labrum to a bone such as a glenoid bone. The suture 114 includes a straight end 116 and a looped end 118, and a thickness which varies from a maximum T_{MAX} at the looped end 118 to a minimum T_{MIN} at the straight end 116. In the illustrated embodiment, the thickness tapers gradually from the apex 119 of the looped end 118 to the tip 120 of the straight end 116. However, in another example, the entire looped end 118 may have a constant thickness T_{MAX}, and the entire straight end 116 (ie. the entire length of the portion extending from the base of the loop 118 to the tip 120) may have a constant thickness T_{MIN}.

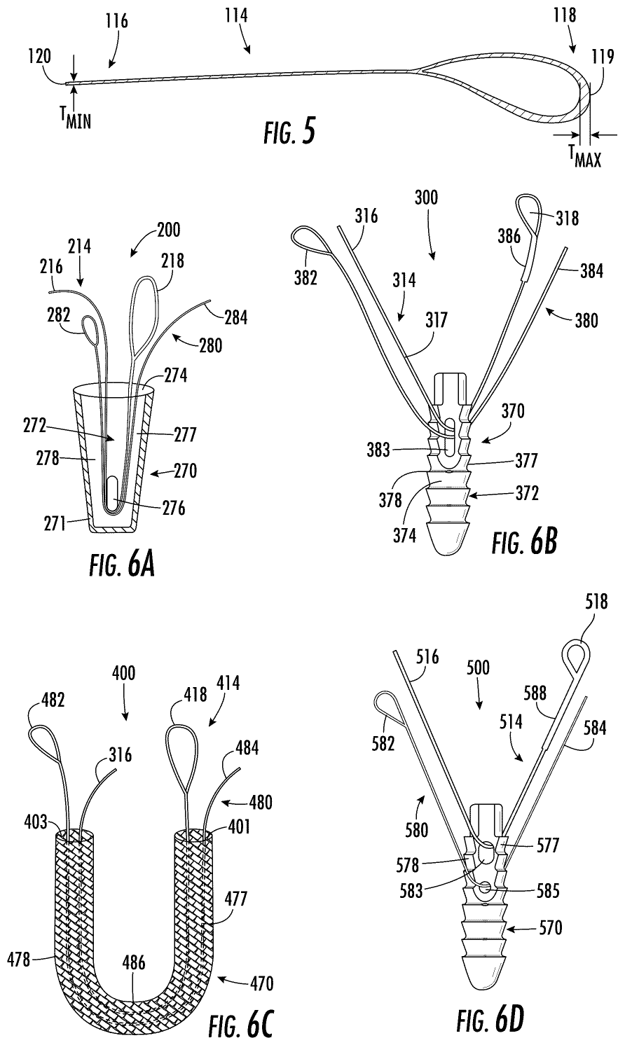

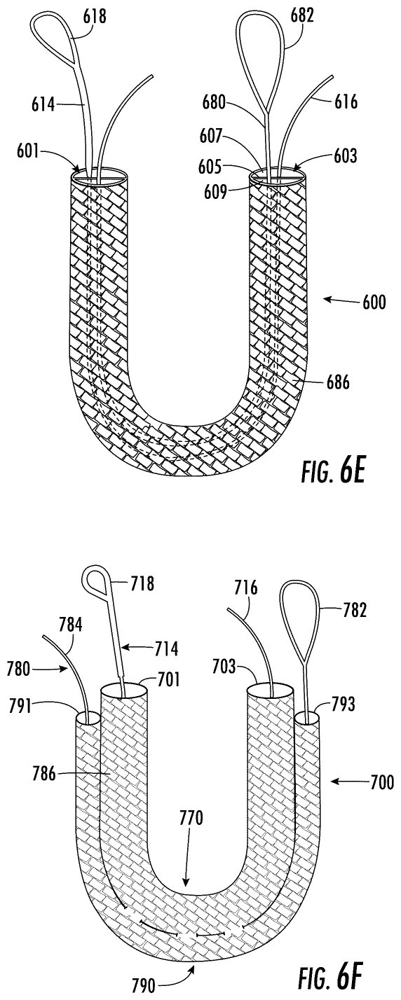

FIG. 6A shows a suture assembly 200 comprising a suture anchor 270, a repair suture 214 similar in structure to the repair suture 114 of FIG. 5, and a shuttle suture 280. The suture anchor 270 comprises an inner wall 271 defining a cannulation 272, an open proximal end 274 communicating with the cannulation 272, and a suture engagement feature such as a rod or suture bridge 276 which extends across the cannulation and divides the cannulation into a first side 277 and a second side 278.

The repair suture 214 has been doubled on itself and positioned in the cannulation 272 such that its looped end 218 extends in a proximal direction along the first side 277 of the cannulation 272 and outwardly through the open proximal end 274, and its tail 216 extends in a proximal direction along the second side 278 of the cannulation 272 and outwardly though the open proximal end 274.

The shuttle suture 280 has a looped end 282 and a tail 284, and has been doubled on itself and positioned in the cannulation 272 such that its looped end 282 extends in a proximal direction along the second side 278 of the cannulation 272 and outwardly through the open proximal end 274, and its tail 284 extends in a proximal direction along the first side 277 of the cannulation 272 and outwardly though the open proximal end 274. The looped end 282 of the shuttle suture 280 is smaller than the looped end 218 of the repair suture 214. In addition, the shuttle suture 280 is thinner than the repair suture 214 throughout all or most of its length.

FIG. 6B shows an alternate suture assembly 300 including a labral suture anchor 370, a repair suture 314, and a shuttle suture 380. The repair suture 314 includes a looped first end 318, a second end 316, and an intermediate section extending between the first and second ends. The intermediate section includes a proximal portion 386 adjacent to the looped first end 316, and a tail portion 317 adjacent to the second end 316. The tail portion 317 is round in cross-section and preferably has a diameter no greater than that of a USP 1 suture (about 0.4 to 0.499 mm), while the proximal portion 386 may have either a round cross-section, with a diameter that is greater than the diameter of the tail portion 317, or a flat, rectangular cross-section with a thickness that is greater than the diameter of the tail portion 317.

The shuttle suture 380, also known as a passing suture, also includes a looped first end 382 and a straight second end 384. As shown here, the shuttle suture 380 has a uniform diameter or thickness through its length. However, in other versions, the passing suture may have a hybrid structure, with a loop and proximal thickened portion formed of tape, and a tail having a round cross-section with a diameter that is less than the thickness of the proximal portion. In any case, the shuttle suture is thinner throughout its length than the repair suture.

As illustrated, the suture anchor 370 includes an anchor body 372 having a ribbed external wall 374 that enhances its engagement with surrounding bone; alternatively, the external wall could include screw threads or other fixation enhancements. In this assembly, the suture engagement feature comprises an elongated oval bore 383 that extends through external wall 374. The looped end 318 of the repair suture 314 extends in a proximal direction alongside and externally of a first side 377 of the anchor body 372, and the tail 316 extends in a proximal direction alongside and externally of a second side 378 of the anchor body 372. The shuttle suture 380 extends in the opposite direction, such that its looped end 382 extends in a proximal direction alongside and externally of the second side 378 of the anchor body 372, and its tail 384 extends in a proximal direction alongside and externally of the first side 377 of the anchor body 372.

FIG. 6C shows an alternate suture assembly 400 including a soft, all-suture anchor 470, rather than the hard anchor 270 of FIG. 6A. The repair suture 414 and shuttle suture 480 are woven through or embedded in a collapsible tube or sleeve 486 which is made from the same material as, and may be integral with, the repair suture 414 and shuttle suture 480. The sleeve 486 is bent into a U-shape having a first side 477, a second side 478, and two proximal openings 401, 403. The looped end 418 of the repair suture 414 extends through the first side 477 of the U-shaped sleeve 486 and out the first proximal opening 401, and the straight end 416 of the repair suture 414 extends through the second side 478 of the U-shaped sleeve and out the second proximal opening 403. The looped end 482 of the shuttle suture 480 extends through the second side 478 of the U-shaped sleeve 486 and out the second proximal opening 403, and the tail 484 of the shuttle suture 480 extends through the first side 477 of the U-shaped sleeve 486 and out the first proximal opening 401.

FIG. 6D shows a double-bore labral suture assembly 500 which is similar to the suture assembly 200 shown in FIG. 6B, except that the repair suture 514 and shuttle suture 580 extend through two separate bores 583, 585, rather than a single bore. The repair suture 514 extends through the proximal bore 583 of the anchor 570, with its looped end 518 extending proximally alongside and externally of a first side 577 of the anchor 570 and its tail end 516 extending proximally alongside and externally of a second side 578 of the anchor 570. The shuttle suture 580 extends in the opposite direction through the distal bore 585, with its looped end 582 extending alongside and externally of the second side 578 and its tail 584 extending alongside and externally of the first side 577. The proximal bore 583 is oval in cross-section and larger than the distal bore 585, which is round in cross-section and preferably has a diameter approximately equal to the diameter of the thickened portion 588 of the repair suture 514. An advantage of this type of double-bore arrangement is that the repair and shuttle sutures 514, 580 are kept separate from one another, reducing the likelihood of tangling.

The all-suture assembly 600 of FIG. 6E is similar to the all-suture assembly 400 of FIG. 6C, except that a partition 605 divides the interior of the tube 686 into two separate channels 607 and 609. The partition 605 is offset from the center of the tube, so that the first channel 607 is larger than the second channel 609. The repair suture 614 passes through the first channel 607 with its looped end 618 extending out a first proximal opening 601 of the tube 686 and its tail 616 extending through a second proximal opening 603 of the tube 686. The shuttle suture 680 extends through the second channel 609, with its looped end extending out the second proximal opening 603 and its tail end extending out the first proximal opening 601.

FIG. 6F shows an alternate suture assembly 700 comprising two all-suture anchors 770, 790 of the same type as all-suture anchor 470 in FIG. 6C, wherein the first anchor 770 is spliced, fused, stitched, or otherwise attached at its base to the second anchor 790, which may be smaller in diameter than the first anchor 770. The repair suture 714 extends through the tube 786 of the first anchor 770, with its looped end 718 extending out a first proximal opening 701 of the tube 786 and its tail 716 extending out a second proximal opening 703 of the tube 786. The shuttle suture 780 extends in an opposite direction through the tube 796 of second anchor 790, with its looped end 782 extending out a second proximal opening 793 of the tube 796 and its tail 784 extending out a first proximal opening 791 of the tube 796.

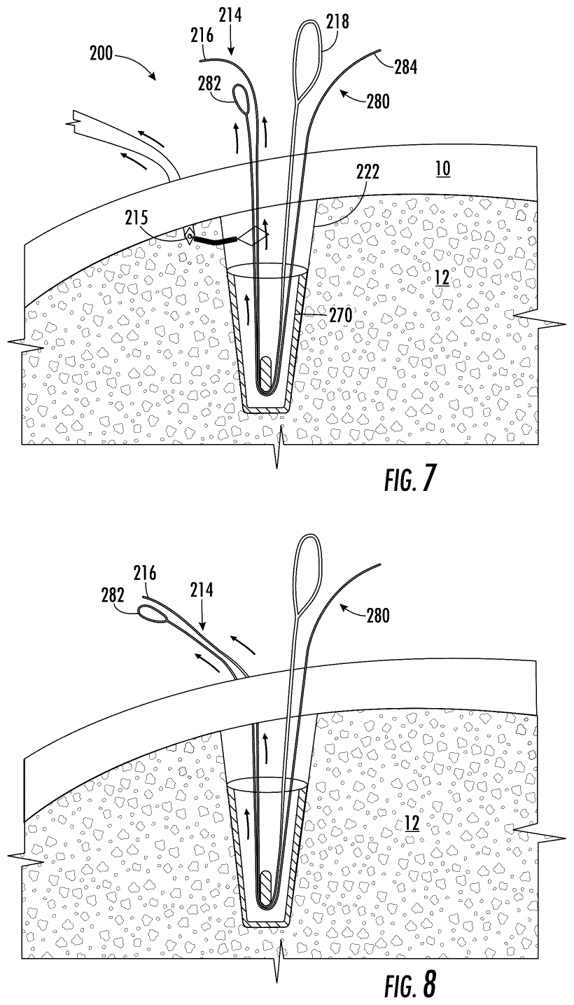

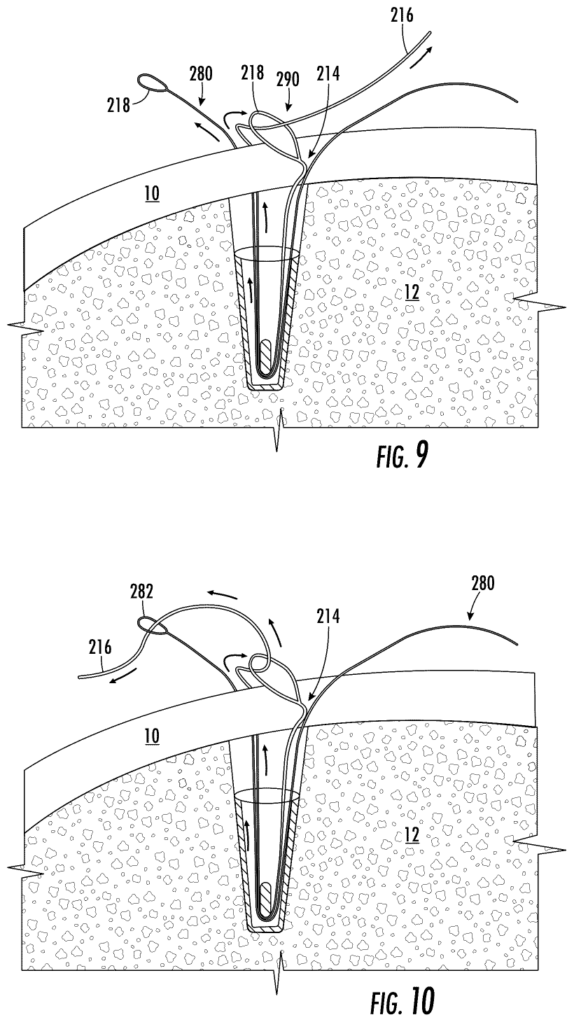

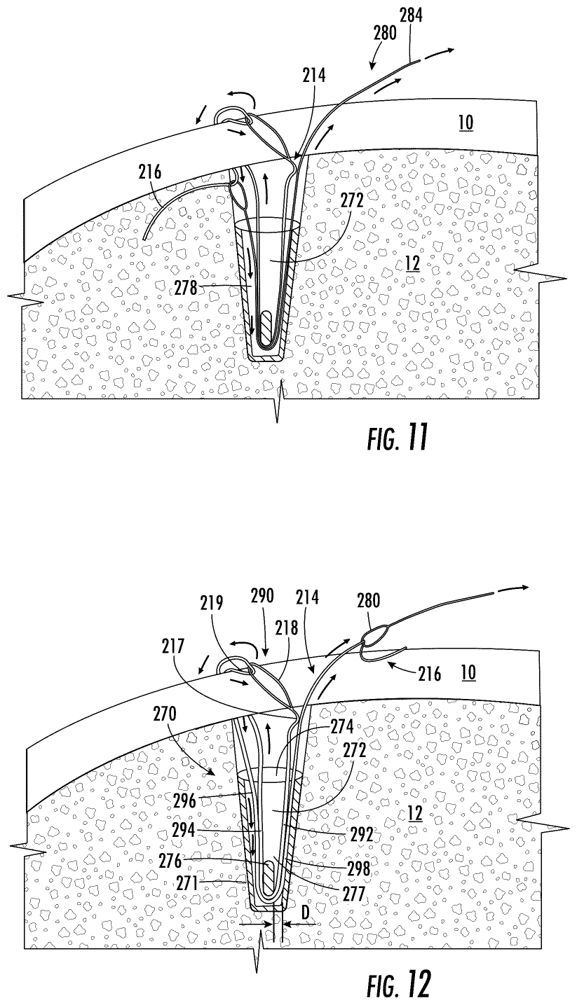

FIGS. 7-12 show a technique for securing soft tissue 10 such as a torn shoulder labrum to a bone 12 such as a glenoid bone using the suture assembly 200 of FIG. 6A. Initially, the suture anchor 270 is inserted in a predrilled socket 222 in the bone 12 at a location to one side of the tissue 10, and a suture passing device 215 is used to pass the tail 216 of the repair suture 214 and the loop 282 of the shuttle suture 280 between the tissue 10 and the bone 12, to the side of the tissue 10 opposite the suture anchor 270, as shown in FIGS. 7 and 8. The tail 216 of the repair suture 214 is then passed back over the tissue 10 and through the looped end 218 of the repair suture to form a luggage tag configuration 290, as shown in FIG. 9. Next, the tail 216 of the repair suture 214 is doubled over itself and drawn through the looped end 282 of the shuttle suture 280, as shown in FIG. 10. The tail 284 of the shuttle suture 280 is then pulled in a proximal direction, drawing the tail 216 of the repair suture 214 down into the second side 278 of the cannulation 272 of the suture anchor, as shown in FIG. 11. Continued pulling of the shuttle suture 280 eventually draws the tail 216 of the repair suture 214 up through the first side 277 of the cannulation 272, and out through the open proximal end 274 of the suture anchor 270, thus tightening the luggage tag configuration 290 against the tissue 10, as shown in FIG. 12. At this point, the shuttle suture 280 may be removed.

In the configuration shown in FIG. 12, the tail 216 of the repair suture 214 can be thought of as comprising four segments: a first segment 292 extending between the base 217 of the loop 218 and the distal end of the suture bridge 276; a second segment 294 extending between the distal end of the suture bridge 276 and the apex 219 of the loop 218 on the second side 278 of the suture bridge 276; a third segment 296 extending between the apex 219 of the loop 218 and the distal end of the suture bridge 276 on the first side 277 of the suture bridge 276; and a fourth segment 298 extending between the distal end of the suture bridge 276 and the looped end 282 of the shuttle suture 280.

The luggage tag configuration 290 is locked into place against the tissue 10 by trapping the segments 292, 294, 296, 298 of the tail 216 of the repair suture 214 in an interference fit between the suture bridge 276 and the inside wall 271 of the suture anchor 279. In order to create this interference fit, the sum of the thicknesses of the first and fourth segments 292, 298 and/or the sum of the thicknesses of the second and third segments 294, 296 should be approximately equal to the distance D between the suture bridge 296 and the inside wall 271 of the suture anchor 279. In the case where the suture tail 216 has a uniform thickness T_{Min} throughout its length and the suture bridge 276 is exactly centered in the cannulation 272, this would mean D should be approximately equal to 2T_{Min}.

In addition to the shoulder labral repairs described above, the methods and devices disclosed herein may be used in any type of surgery involving the attachment of tissue to tissue. This includes soft-tissue-to soft-tissue repairs, soft-tissue-to-hard-tissue (bone) repairs, or even certain types of hard-tissue-to-hard-tissue (bone-to-bone) attachments.

Variations of the method of FIGS. 7-12 can also be performed using double-bore suture anchors such as that shown in FIG. 6D, or all-suture anchors such as those shown in FIGS. 6C, E, and F rather than the single-bore, hard suture anchor of FIG. 6A. In single- or double-bore suture anchors, the luggage tag configuration is locked in place by an interference fit between the sutures and the surrounding bore walls, and in all-suture anchors, the locking is caused by contact force exerted by the all-suture anchor on the surrounding bone when the anchor is deployed, rather than by an interference fit between the sutures and an internal suture bridge.

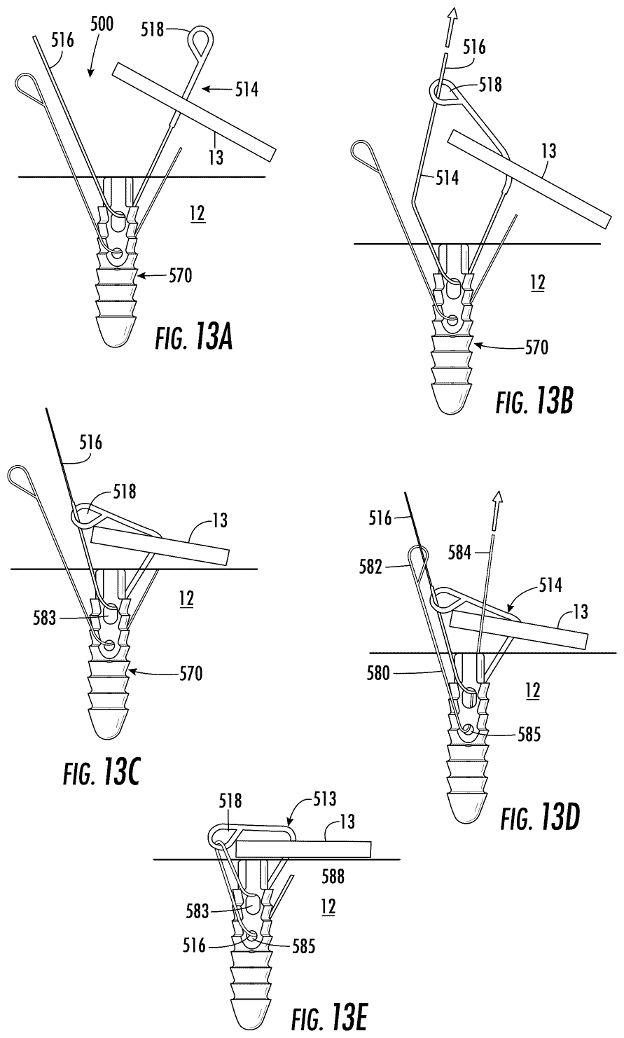

FIGS. 13A-E show a method of repairing tissue using the double-bore labral suture assembly 500 of FIG. 6D. Initially, the anchor 570 is inserted into bone 12 and the looped end 518 of the repair suture 514 is drawn through a flap of tissue 13 overlying the bone 12, as shown in FIG. 13A. Next, the tail 516 of the repair suture 514 is inserted through the looped end 518 and pulled in a proximal direction, as shown in FIG. 13B. This causes the repair suture 514 to slide through the proximal bore 583, creating tension which pulls the tissue 13 distally, towards the bone 12, as shown in FIG. 13C. The tail 516 of the repair suture 514 is then inserted through the looped end 582 of the shuttle suture 580, as shown in FIG. 13D, and the tail 584 of the shuttle suture 580 is pulled in a proximal direction. This draws the looped end 582 of the shuttle suture 580 through the second bore 585 in the anchor 570, pulling the tail 516 of the repair suture 514 along with it. Continued proximal movement of the shuttle suture 580 causes both the looped end 518 of the repair suture 514 and the tissue 13 to move distally, until the tissue 13 contacts the bone 12, as shown in FIG. 13E. At this point, the thickened portion 588 of the looped end 518 of the repair suture 513 is trapped in an interference fit with the side walls of the distal bore 585, locking the repair 513 in place so that slippage does not occur.

FIGS. 14A-C show various single-bore labral anchor configurations. The labral anchor 370 of FIG. 14A, also shown in FIG. 6B, includes a ribbed anchor body 372 having an elongated oval bore or slot 383 formed near its proximal end 399. The length of the bore 383 is preferably slightly greater than the combined widths or diameters of the shuttle suture 380 and the tail 316 of the repair suture in FIG. 14 in order to allow the sutures to easily pass through the bore 383 during the repair. The width of the bore 383 is approximately equal to the width or diameter of the thickened portion 386 of the repair suture 314, in order to create an interference fit between the thickened portion 386 and the bore 383 after the repair has been completed.

The labral anchor 870 of FIG. 14B includes a ribbed anchor body 872 having a round bore 883 formed near its proximal end 800. The diameter of the bore 883 is preferably greater than the combined widths or diameters of the shuttle suture and the tail of the repair suture with which it is used in order to allow the sutures to easily pass through the repair, but approximately equal to the width or diameter of the thickened portion of the repair suture in order to create an interference fit between the thickened portion and the bore 883 after the repair has been completed.

The labral anchor 970 of FIG. 14C includes a ribbed anchor body having a teardrop shaped bore 983 formed near its proximal end 999. The bore 383 has a relatively wide distal end 995 and a narrow proximal end 997. The width of the distal end 995 of the bore 383 is preferably greater than the combined widths or diameters of the repair and shuttle suture with which it is used so that the sutures can easily pass through during the repair. The width of the proximal end 997 is preferably equal to, or slightly less than, the width or diameter of the thickened portion of the repair suture with which it is used, so that when the repair is completed, the repair suture can be pulled upwardly into locking engagement with the proximal end 997 of the bore 983.

FIGS. 15A-F show various double-bore labral anchor configurations. The labral anchor 1070 FIG. 15A is substantially the same as the labral anchor 370 of FIG. 14A, except that two elongated oval bores 1083, 1085 are provided—one for accommodating a repair suture and another for accommodating a shuttle suture. In the illustrated examples the two oval bores 1083, 1085 have equal dimensions; however, in some cases it may be desirable for one bore to be larger than the other.

The labral anchor 1170 shown in FIG. 15B includes an oval proximal bore 1183 of the type shown in FIG. 14A and a round distal bore 1185 of the type shown in FIG. 14B.

The labral anchor 1270 shown in FIG. 15C includes a round proximal bore 1283 of the type shown in FIG. 14B and an oval distal bore 1285 of the type shown in FIG. 14A.

The labral anchor 1370 shown in FIG. 15D includes a pair of teardrop shaped bores 1383, 1385 of the type shown in FIG. 14C. In the illustrated example, the two bores 1383, 1385 have the equal dimensions; however, in some cases it may be desirable for one bore to be larger than the other.

The labral anchor 1470 shown in FIG. 15E includes a teardrop shaped proximal bore 1483 of the type shown in FIG. 14C and a round distal bore 1485 of the type shown in FIG. 14B.

The labral anchor 1570 of FIG. 15F includes a round proximal bore 1583 of the type shown in FIG. 14B and a teardrop shaped distal bore 1585 of the type shown in FIG. 14C.

In some cases, it may be desirable to provide any of the suture anchors 15A-F with a third bore so that the repair suture may be locked inside the anchor at two locations, thus reducing the likelihood of slippage even further.

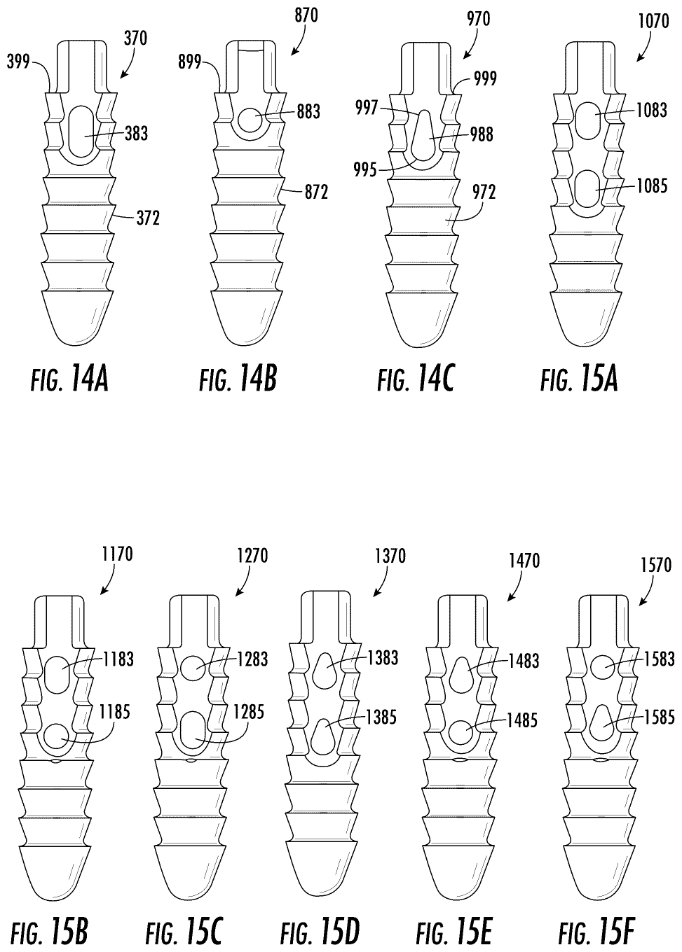

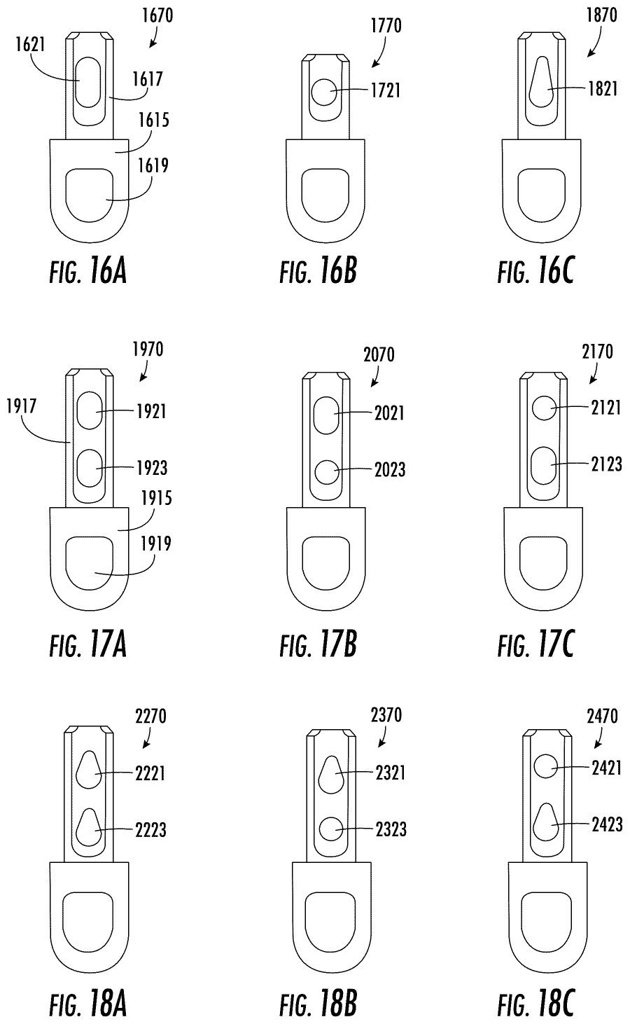

FIGS. 16A-C show various single-bore distal tip configurations for lateral suture anchors. Tip 1670 shown in FIG. 16A includes a distal portion 1615 designed to be inserted into bone and a proximal neck 1617 designed to extend into the inner cannulation of a proximal anchor body. The distal portion 1615 includes an eyelet 1619 for receiving repair sutures from a medial row. The neck 1617 includes an elongated oval bore or slot 1621 for receiving a pair of stay sutures that may be used after a repair procedure to augment the strength of a repair after the tip 1670 has been placed, and to prevent or reduce and stabilize dog-ear lesions which might otherwise interfere with healing. The distal tip 1670 may be used for a variety of tissue fixation repairs, including the repairs described in connection with 5A-D, 6A-C, and 11A-E of U.S. Pat. No. 11,350,926B1 to Ninh, the entire contents of each of which are incorporated herein by reference.

The tip 1770 shown in FIG. 16B is similar to the tip 1670 of FIG. 16a, but the bore 1721 for receiving the stay sutures is round rather than oval.

The tip 1870 shown in FIG. 16C includes a teardrop shaped bore 1821 similar to the teardrop shaped bore of FIG. 14C.

FIGS. 17A-F show a variety of double-bore distal tip configurations for lateral suture anchors which may be used for the same types of repairs as the single-bore configurations shown in FIGS. 16A-C. Each of the distal tips shown in FIGS. 17A-F is part of a two-piece design including a proximal anchor body which may be similar in structure to the proximal anchor body 36 of the lateral suture anchor shown in FIGS. 2A and B, or may resemble the proximal anchor body of various other lateral suture anchors that are commercially available.

The tip 1970 shown in FIG. 17A includes a distal portion 1915 designed to be inserted into bone and a proximal neck 1917 designed to extend into the open end of a tubular cylinder driver. The distal portion 1915 includes an eyelet 1919 for receiving repair sutures from a medial row. The neck 1917 includes a pair of elongated oval bores or slots 1621, 1623, each of which may receive a stay suture.

The tip 2070 shown in FIG. 17B includes an elongated oval proximal bore 2021 and a round distal bore 2023.

The tip 2170 shown in FIG. 17C includes a round proximal bore 2121 and an elongated oval distal bore 2123.

The tip 2270 shown in FIG. 17D includes a pair of teardrop shaped bores 2221 and 2223.

The tip 2370 shown in FIG. 17E includes a teardrop shaped proximal bore 2321 and a round distal bore 2323.

The tip 2470 shown in FIG. 17F includes a round proximal bore 2421 and a teardrop shaped distal bore 2423.

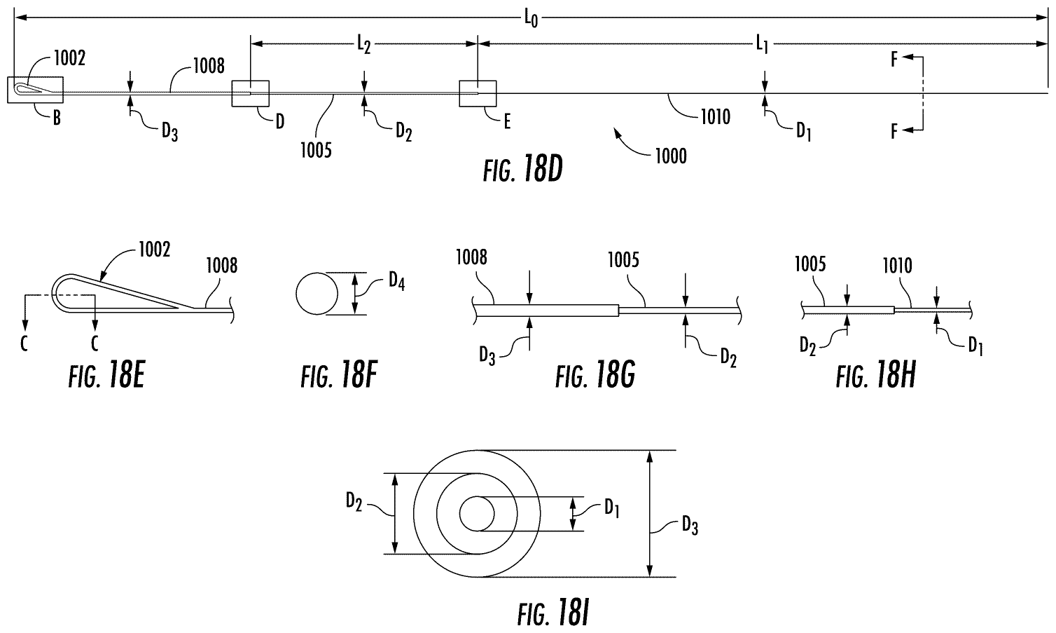

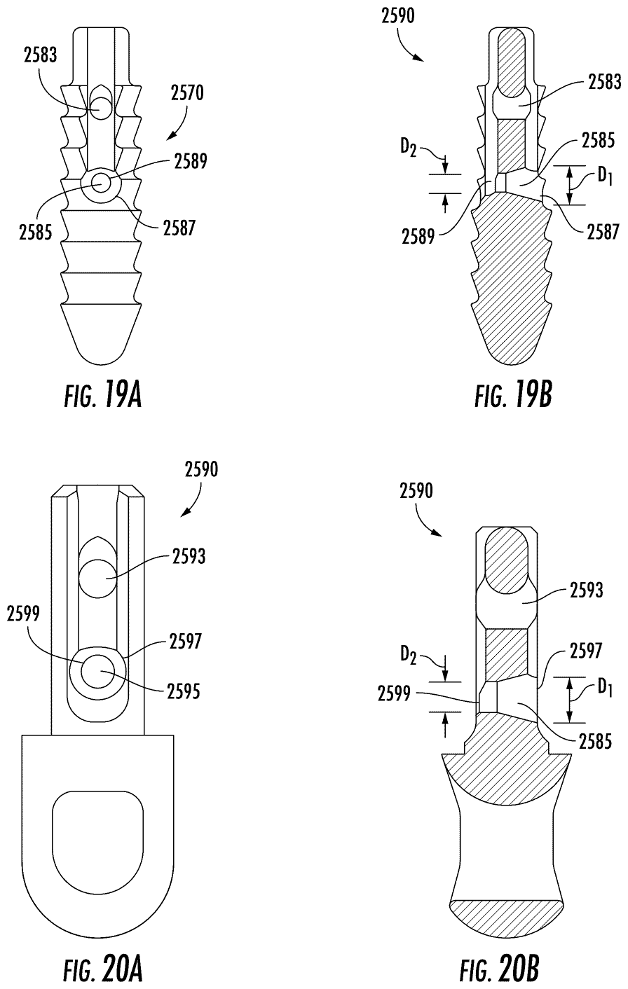

FIGS. 18A-F show an alternate loop-and-tail suture 1000 usable in any of the suture anchors shown in FIGS. 6B-F, 14A-C, 15A-F, 16A-C, and 17A-F, and in the methods shown in FIGS. 7-12 and 13A-E, as described above. The suture 1000 is braided and has a round cross-section throughout its length L. It includes a loop 1002, a tail 1010, a proximal portion 1008, and a transition portion 1005 extending between the tail 1010 and the proximal portion 1008. The suture 1000 has a minimum diameter D₁ in its tail 1010, which has a length L₁ equal to about 50% to about 75% of the entire length of the suture L₀. The transition portion 1005 has a diameter D₂ that is greater than the D₁, and a length L₂ that is equal to about 10% to about 40% of the length of L₁. The proximal portion 1008 has a diameter D₃ which is larger than the diameter D₂ of the intermediate portion 1006. The cross-sectional diameter D₄ of the loop 1002 is less than the diameter D₃ of the proximal portion 1008, and larger than the diameter D₁ of the end portion D₁. In a preferred embodiment, D₁ is equal to the diameter of a USP size 0 suture; D₂ is equal to the diameter of a USP size 1 suture, and D₃ is equal to the diameter of a USP size 2 suture. The small diameter of the tail 1010 makes the suture 1000 easy to manipulate, while the wider transition and proximal portions 1005, 1008 add strength, and enhance the ability of the suture 1000 to lock into place within a properly constructed suture anchor.

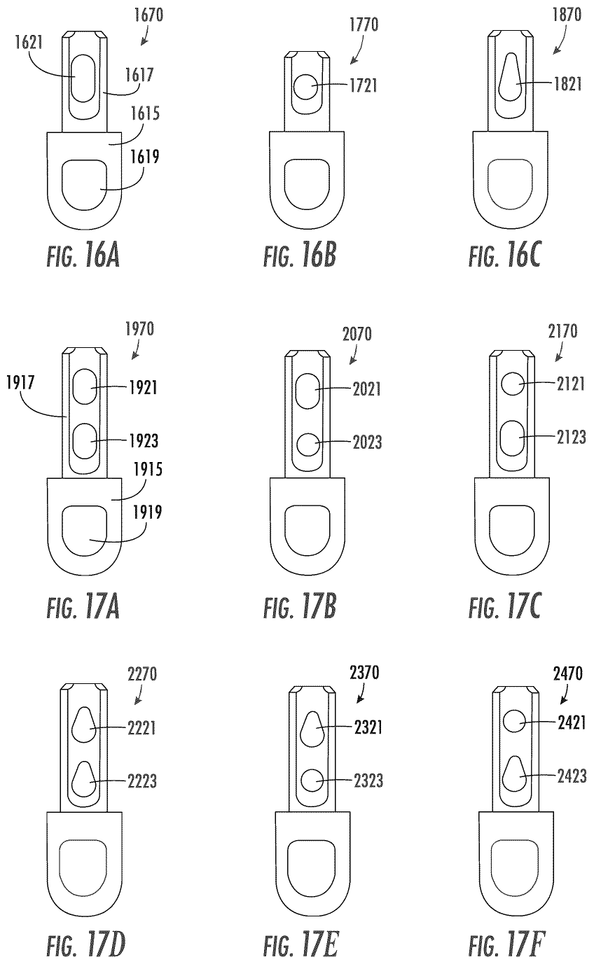

FIGS. 19A and B show a labral anchor suture tip 2570 similar to the tips shown in FIGS. 15A-F, except that both the proximal bore 2583 and the distal bore 2585 are round. The distal bore 2585 is chamfered or countersunk so that it extends between a front opening 2587 having a first diameter D₁ and a rear opening 2589 having a second diameter D₂ that is less than D₁. The second diameter D₂ is approximately equal to the diameter of a USP 2 suture, thus creating an interference fit with the proximal portion of the suture of FIGS. 18A-F when in the configuration shown in FIG. 13E. This allows the suture to be locked securely in place, minimizing the likelihood of slippage.

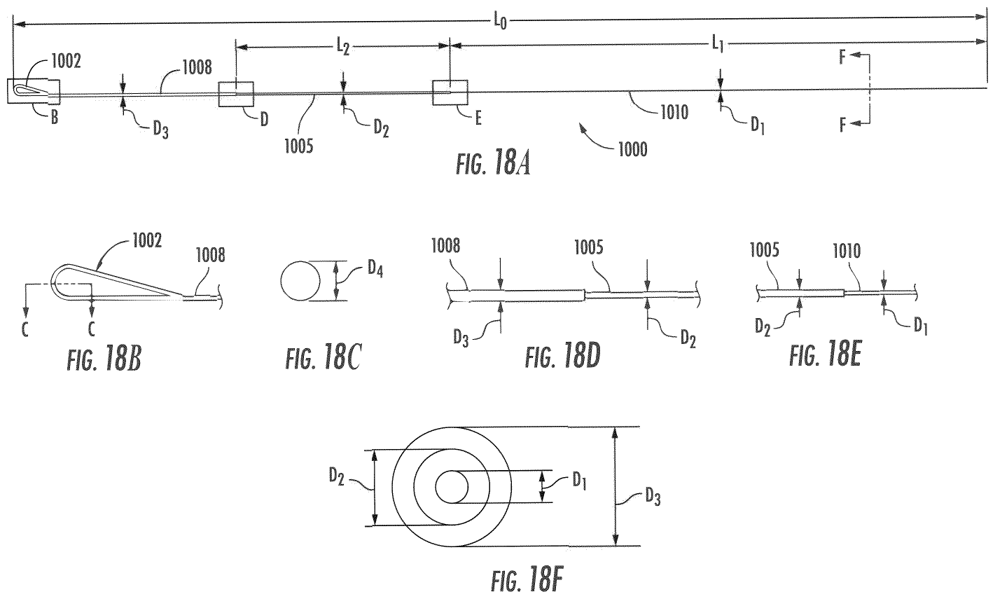

FIGS. 20A and B show a lateral row anchor suture tip 2590 which, like the suture tip 2570 of FIGS. 19A and B, includes two round bores 2593, 2595, wherein the distal bore 2595 extends between a front opening 2597 having a first diameter D₁ and a rear opening 2599 having a second diameter D₂ that is less than D₁. Once again, the second diameter D₂ approximately equal to the diameter of a USP 2 suture, in order to create an interference fit with the proximal portion of the suture of FIGS. 18A-F.

While embodiments of the invention have been illustrated and described, it is not intended that these embodiments illustrate and describe all possible forms of the invention. Rather, the words used in the specification are words of description rather than limitation, and it is understood that various changes may be made without departing from the spirit and scope of the invention.

What is claimed is:

1. A suture comprising:
a first end including a loop; and
a straight linear tail, the tail including
a proximal portion adjacent the loop, and
an end portion that is thinner than the proximal portion.

2. The suture according to claim 1, wherein the suture is braided.

3. The suture according to claim 1, wherein the loop and tail have round cross-sections with predetermined diameters.

4. The suture according to claim 3, wherein the cross-sectional diameter of the loop is less than the cross-sectional diameter of the proximal portion of the loop.

5. The suture according to claim 3, wherein the tail comprises three distinct sections including:
the proximal portion;
the end portion; and
and intermediate portion between the proximal portion and the end portion.

6. The suture according to claim 5, wherein;
the end portion has a first cross-sectional diameter D₁;
the intermediate portion has a second cross-sectional diameter D₂ that is greater than D₁; and
the proximal portion has a third cross-sectional diameter D₃ that is greater than D₂.

7. The suture according to claim 6, wherein the cross-sectional diameter of the loop equals D₂.

8. The suture according to claim 7, wherein the diameter D₃ of the proximal portion is twice the diameter D₂ of the intermediate portion.

9. The suture according to claim 5, wherein the suture has a predetermined length, and the end portion has a predetermined length that is 50% to about 75% of the length of the suture.

10. The suture according to claim 9, wherein the intermediate portion has a predetermined length that is about 10% to about 40% of the length of the end portion.

11. A suture anchor for implanting a suture having a thickened portion with a predetermined diameter or thickness, the suture anchor comprising:
a distal passageway configured to receive the suture, the passageway having
a rear opening with a dimension selected to create an interference fit with the thickened portion of the suture, and
a front opening which is larger than the rear opening.

12. The suture anchor according to claim 11, wherein:
the front and rear openings are round; and
the rear opening has a diameter approximately equal to the diameter or thickness of the suture.

13. The suture anchor according to claim 10, further comprising a proximal passageway configured to receive a second suture.

14. A suture assembly comprising:
a suture anchor having a distal passageway including
a rear opening having a predetermined size, and
a front opening having a predetermined size larger than the rear opening; and
a repair suture extending through the distal passageway, the repair suture having
a first end including a loop; and
a straight linear tail, the tail including
a proximal portion adjacent the loop, the proximal portion having a cross-section with a size approximately equal to the size of the rear opening, and
an end portion that is thinner than the proximal portion.

15. The suture assembly according to claim 14, wherein:
the suture anchor further comprises a proximal passageway, and
the assembly further comprises a passing suture including a loop and a tail, wherein the passing suture extends through the proximal passageway, with the loop of the passing suture disposed on an opposite side of the suture anchor from the loop of the repair suture, and the tail of the passing suture disposed on an opposite side of the suture anchor from the tail of the repair suture.

16. The suture assembly according to claim 14, wherein the tail of the repair suture comprises three distinct sections including:
the proximal portion;
the end portion; and
an intermediate portion between the proximal portion and the end portion.

17. The suture assembly according to claim 16, wherein;
the end portion is round in cross-section and has a first cross-sectional diameter D₁;
the intermediate portion is round in cross-section and has a second cross-sectional diameter D₂ that is greater than D₁; and
the proximal portion is round in cross-section and has a third cross-sectional diameter D₃ that is greater than D₂.

18. The suture assembly according to claim 17, wherein the diameter D₃ of the proximal portion is twice the diameter D₂ of the intermediate portion.

19. The suture assembly according to claim 16, wherein the suture has a predetermined length, and the end portion has a predetermined length that is 50% to about 75% of the length of the suture.

20. The suture assembly according to claim 19, wherein the intermediate portion has a predetermined length that is about 10% to about 40% of the length of the end portion.
